(19) United States
(12) Patent Application Publication
(10) Pub. No.: US 2015/0327777 A1
(43) Pub. Date: Nov. 19, 2015

(54) TISSUE MONITORING APPARATUS AND SYSTEM

(71) Applicant: Stryker Corporation, Kalamazoo, MI (US)

(72) Inventors: Marko N. Kostic, Portage, MI (US); Christopher John Hopper, Kalamazoo, MI (US); Patrick Lafleche, Kalamazoo, MI (US); Scott Lawrence, Kalamazoo, MI (US)

(21) Appl. No.: 14/708,383

(22) Filed: May 11, 2015

Related U.S. Application Data
(60) Provisional application No. 61/993,099, filed on May 14, 2014.

(51) Int. Cl. A61B 5/0205; A61B 5/024; A61F 7/00; A61B 5/1455; A61B 5/026; A61M 1/00; A61B 5/021; A61B 5/1459

(52) U.S. Cl. CPC A61B 5/0205; A61B 5/021; A61B 5/024; A61B 5/02055; A61B 5/1459; A61B 5/14552; A61B 5/1455; A61B 5/0261; A61M 1/0031; A61F 7/00; A61M 2205/3303; A61M 2205/3306; A61M 2230/20; A61F 2007/0094

(57) ABSTRACT

A tissue monitoring device for selecting, administering or adjusting a treatment based on information obtained by monitoring the tissue. The device includes a substrate for applying to the tissue, for example, a human or an animal external, or internal tissue, including vascular tissue; a light source supported by the substrate, the light source emitting light of a first frequency for directing the emitted light onto a surface of the tissue; a photodetector carried by the substrate spaced from the light source for detecting scattered light, wherein the scattered light is the emitted light scattered by the tissue; and a controller in communication with the photodetector and the light source.

This application claims the benefit of U.S. Provisional Patent Application No. 61/993,099, filed on May 14, 2014 which is incorporated herein by reference in its entirety and is commonly owned by Stryker Corporation of Kalamazoo, Mich.

### TECHNICAL FIELD AND BACKGROUND OF THE INVENTION

The present invention relates to monitoring a condition of a subject (e.g. person or animal), including the response of the subject to treatment, in order to assess the person's condition or adjust the treatment based on the response of the subject.

When a person or animal (hereinafter referred to as “subject”) is undergoing treatment, methods of controlling or adjusting the treatment are often based on the subject's vital signs, which typically include body temperature, heart rate, respiratory rate, and blood pressure. However, conventional physiological tests, such as these, may not provide a real-time measure of the condition of a subject or clinical effect of a treatment to a subject, which in some instances can impact the assessment of the subject and/or the efficacy of the treatment.

### SUMMARY OF THE INVENTION

Accordingly, a tissue monitoring apparatus or system is described that is configured to monitor internal or external tissue, and to collect data from the tissue, which may be used to determine one or more parameters about the tissue state, for example, changes in tissue perfusion or changes in tissue metabolism, in order to provide information, including feedback information, which may provide a more complete clinical picture about the subject's condition, including efficacy of a specific treatment.

In one embodiment, a method of monitoring tissue includes a light source emitting light of a first frequency, directing the emitted light onto a surface of tissue, and detecting scattered light with a photodetector, wherein the detected scattered light is the emitted light scattered by the tissue. For example, the method may include monitoring the effects of an internal or external factor on a tissue.

In one aspect, the method further includes determining the scattered light intensity of the detected scattered light with respect to the emitted light.

In another aspect, emitted light is directed from more than one light source. Further, emitted light with a second frequency different from the first frequency may be directed on to the tissue.

In any of the above, the identity of each light source is associated with detected scattered light.

In any of the above, emitted light is directed from one or more of an LED, a laser, or a plasma lamp. Further, in any of the above, the light source may comprise a contiguous light source.

Further, information about the scattered light intensity may be transmitted, for example, wirelessly to a remote device.

In addition, any of the above tissue monitoring methods may be employed in monitoring changes in status in intracellular and extracellular elements of the tissue.

In yet another aspect, a treatment may be selected, administered, or adjusted based on the information obtained from the monitored tissue.

In another embodiment, a tissue monitoring device includes a substrate for applying to an external or internal tissue of a subject (e.g. human or an animal). A light source is supported by the substrate and emits light of a first frequency for directing the emitted light onto a surface of the tissue. A photodetector is carried by the substrate spaced from the light source for detecting scattered light, wherein the scattered light is the emitted light scattered by the tissue. The device further includes a controller in communication with the photodetector and the light source. The substrate may comprise a transfer film, a stent, or may comprise a treatment device.

In any of the above devices, the controller determines the scattered light intensity with respect to the intensity of the emitted light.

In any of the above devices, the controller may determine the scattered light intensity as a function of time.

In any of the above devices, the light source may include more than one light source, with each light source spaced from the photodetector by different distances. For example, the light source may include one or more of an LED, a laser, or a plasma lamp. Further, the light source may emit light at a second frequency, wherein the second frequency is different from the first frequency.

In other aspects, the light source comprises a discrete light source or a contiguous light source.

In any of the above devices, each light source has an identity, and the controller correlates the identity with the detected scattered light intensity.

According to any of the above devices, the photodetector comprises one or more photosensitive elements, such as a photodiode, a phototransistor, a photomultiplier, or a CCD.

In yet other aspects, according to any of the above devices, the substrate comprises a dielectric material.

According to yet other aspects, a wireless module is carried by the substrate, with the controller in communication with the wireless module.

In any of the above devices, the controller is configured to monitor changes in status in intracellular and extracellular elements of the tissue.

According to yet another embodiment, a tissue monitoring system includes a tissue monitoring device, such as any of the tissue monitoring devices described above, and a remote device, such as a treatment device. Optionally, the system may also include a display. Further, the system may include a controller for collecting information about the tissue, a data storage device, and/or a device for further analyzing information collected by the controller.

In one aspect, the remote device comprises a treatment device for applying a treatment to a subject, and with the controller of the tissue monitoring device in communication with the treatment device for adjusting the treatment based on the scattered light intensity detected by the photodetector.

In any of the above, the remote device may include a display in communication with the controller that displays information based on the scattered light intensity detected by the photodetector.

In any of the above, the remote device may include a data storage unit in communication with the controller for storing information based on the scattered light intensity detected by the photodetector.

In any of the above, the remote device may comprise or further comprise a remote controller, with the remote controller in communication with the local controller for analyzing the scattered light detected by the photodetector.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTIONS OF THE DRAWINGS

FIG. 1 is a schematic view of a tissue monitoring system;

FIG. 2 is a schematic view of a tissue monitoring device applied to tissue;

FIG. 2A is a graph illustrating the Absorption Spectra of Oxygenated (HbO2) and Deoxygenated (HHb) Hemoglobin;

FIG. 3 is an illustration of the operation of the tissue monitoring device;

FIG. 4 is an illustration of the tissue monitoring device located on a person's neck; and

FIG. 5 is an illustration of the underlying vasculature that may be located underneath the tissue monitoring device when located on a person's neck.

### DETAIL DESCRIPTION OF PREFERRED EMBODIMENTS

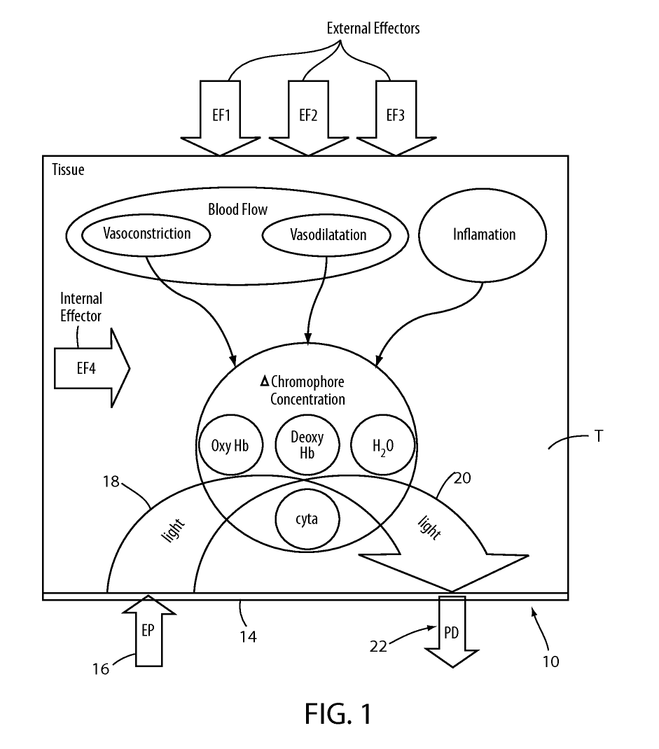

Referring to FIG. 1, the numeral 10 generally designates an apparatus for monitoring a change or changes in tissue. As will be more fully described below, apparatus 10 is configured to monitor changes in tissue to assess the condition of a subject, including the clinical effect of a treatment on a subject. The type of conditions or treatments may vary and include, for example, thermal conditions, brain perfusion, heart conditions, trauma, physical interface, such as pressure, temperature management therapy, drug therapy, wound treatment, or surgery.

Apparatus 10 is configured to measure the tissues response to external and/or internal effecters. External effecters include, for example, temperature, force, and chemicals. Internal effecters include, for example, pharmacological compounds (e.g. drugs), infection, or acts upon the tissue causing specific physiological responses. For example, physiological responses may include tissue responses from positive or negative pressure, which may include vasoconstriction, vasodilation or inflammation. Positive pressure may be applied to when a person is supported on a surface, such as a mattress or a pad. Negative pressure may be applied, for example, when wounds are treated with negative pressure. These physiological responses result in concentration changes of specific tissue chromophores (e.g., oxygenated and deoxygenated hemoglobin, water, cytochrome α). The rate at which these changes occur can provide an indication of the severity of the condition.

For example, the intensity of the inflammation process is usually proportional to the degree of tissue injury. When certain infections occur, inflammation can develop very rapidly. The body's response to inflammation is to enlarge macrophage cells followed by mobilization of the macrophage cells to provide a first line of defense. The second line of defense is the invasion of neutrophil cells. In each case, the migration of such cells provides valuable insight into a condition of a tissue. Similarly, changes in concentration of other tissue chromophores, can provide valuable information about the subject's response to treatment.

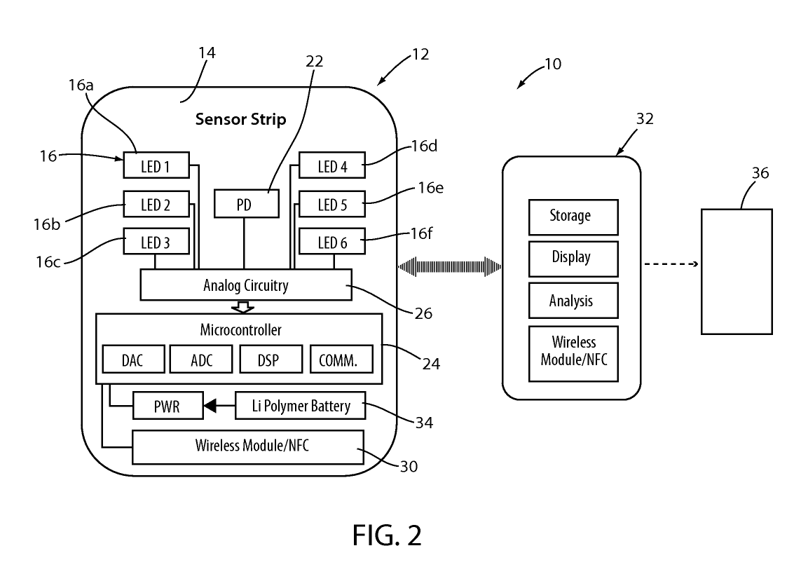

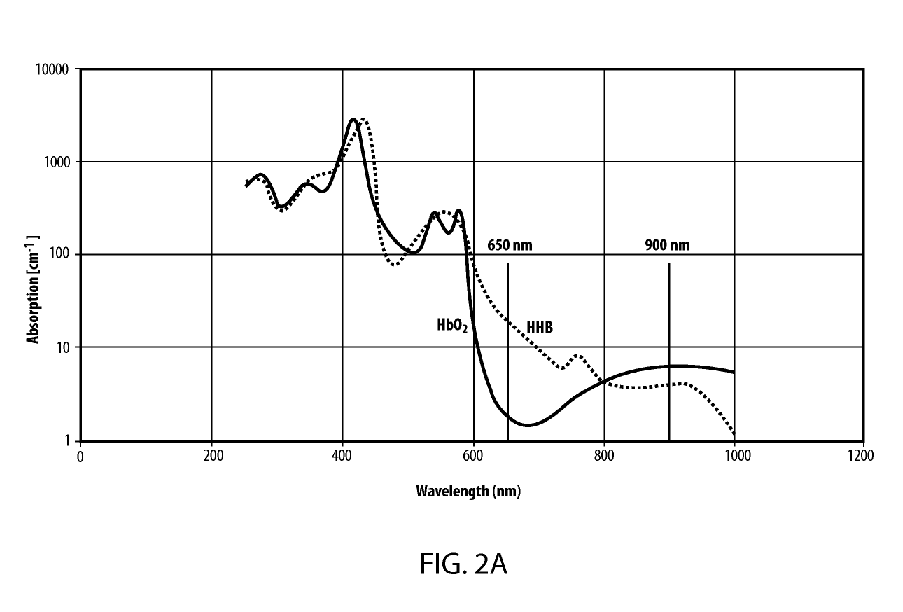

Referring to FIG. 2, apparatus 10 includes a sensor assembly 12, which is configured to emit and direct light on tissue, including external tissue or internal tissue, including vascular tissue, and to detect the light scattered by the tissue to measure how the chromophore levels at different depths of the tissue vary.

In the illustrated embodiment, sensor assembly 12 is mounted to a carrier 14 and includes a light source 16, which is also mounted on carrier 14. Carrier 14, for example, may comprise a flexible strip of dielectric material with an optional adhesive layer applied thereto, which allows the assembly to be applied to tissue, for example, when applied to external tissue. Further, carrier 14 may be disposable. As will be described below, a variety of different substrates may be used for carrier 14 and other methods of attachment may be used, especially when applied to internal tissue.

Light source 16 emits light at at least one frequency, for example, between 600 and 1400 nm, which is known as a biologically relevant spectrum. As noted above, light source 16 directs light 18 onto a subject's tissue T through the incident surface. The light is then scattered in the tissue by encountering numerous scatterers that will cause a change in its direction. A fraction of photons is scattered such that these photons exit tissue at the incident surface. The concentration of chromophores of interest along the scattering path will affect photon absorption and, therefore, determine the fraction of initially emitted light that is scattered back at the incident surface.

The scattered light 20 (FIG. 1) is then detected by at least one detector 22, such as a photosensor or photodetector, including a photodiode, a phototransistor, a photomultiplier, a photoresistor, an active pixel sensors (APSs), or a charge coupled device (CCD) or the like, which also may be mounted to carrier 14. Detector 22 detects the scattered light that exits the surface of the tissue and may detect a single frequency or multiple frequencies, including a broad spectrum of frequencies.

In the illustrated embodiment, detector 22 is located adjacent but spaced from light source 16 so that detector 22 detects light exiting from the same surface, though spaced from, light source 16. Detector 22 is then coupled (either directly or wirelessly) to a microcontroller 24 via circuitry 26 (either analog or digital), which also may be mounted to carrier 14. Microcontroller 24 includes a processor and memory, which may store suitable software or applications that are executed by the processor to receive the signals from detector 22 and may analyze the signals to evaluate the physiological changes in the underlying tissue, and thereby together with light source 16 and detector 22 form a tissue monitoring device. Microcontroller 24 may include an analog digital converter (ADC) when circuitry 26 is analog. It should be understood that microcontroller 24 may also be remotely mounted, separate and apart from the tissue monitoring device, with detector 22 coupled to microcontroller 24 wirelessly via a transmitter provided in the tissue monitoring device so that detector 22 can transmit information about the scattered light, for example, the scattered light intensity to microcontroller 24.

Physiological changes in the underlying tissue manifest themselves in changes in concentration of specific tissue chromophores, for example, oxygenated and deoxygenated hemoglobin, water, cytochrome α. These concentration changes are detected via attenuation (i.e. absorption) of chromophore-specific wavelengths of light (i.e. photons) that are sourced by the light source 16. Once the acquired signal is processed by microcontroller 24, the information may then be communicated via a communication device 30, for example, a wireless module, such as a Bluetooth device or a near field communication (NFC) device to a remote device 32. Apparatus 10 may, therefore, form part of a feedback loop for a control system of another device, more fully described below.

To power light source 16, detector 22, and microcontroller 24, apparatus 10 may also incorporate a power supply 34, which may also be mounted to carrier 14. Power supply 34 may comprise a capacitor or an electrochemical cell, such as a lithium polymer battery or the like, and corresponding circuitry.

As noted above, sensor assembly 12 includes a light source 16. Light source 16 may comprise one or more discrete light sources. In different configurations, light sources are contiguous or spaced. Further, each light source may generate light at a single frequency, multiple frequencies, or a broad spectrum of frequencies. For example, the light source may comprise a light emitting device, such as an LED, a laser, or a plasma lamp. In the illustrated embodiment, light source 16 comprises six spaced light sources 16a, 16b, 16c, 16d, 16e, and 16f (e.g. LEDs). Optionally, one group of light sources 16a-16c may generate light at one frequency, for example, in the red segment of the light spectrum, and another group of light sources 16d-16f may generate light at a second, different frequency, such as the near infra-red segment of the light spectrum so that chromophores that absorb the red light can be distinguished from the chromophores that absorb the infrared light. For example, a suitable wavelength for one group may be from about 600-700 nm or about 650 nm, and a suitable wavelength for the other group may be from about 850-950 nm or about 900 nm. At these wavelengths, tissue chromophores are major absorbers of photons. The photon absorption attenuates the detected light and, hence, enables extrapolation of information about clinically-relevant intracellular and extracellular elements of the tissue.

By measuring the scattered light intensity and comparing it to the incident light intensity, microcontroller 24 can measure the amount of attenuation of the scattered light to measure the absorption that accrued in the tissue for each frequency of light, which can then be used to determine the concentrations of chromophores of interest to determine, for example, the oxygenated or deoxygenated hemoglobin, water, or cytochrome α. This information, when monitored continuously or periodically, can then be used by the microcontroller 24 to identify trends in the subject's relative physiological condition, for example, a physiological condition related to disease, illness, injury, or a subject's response to treatment.

Further, as briefly noted above, this information may be forwarded to remote device 32 for storage, display, for example, to a caregiver, further analysis, or for controlling a second remote device, for example, such as a treatment device 36. For example, treatment device 36 may comprise a temperature management device for applying thermal therapy to a subject. Suitable temperature management devices, such as warming devices or cooling devices, are available from Stryker Corporation under the trademark Medi-Therm®. Thermal management systems, such as a Medi-Therm® device from Stryker, may be used by a caregiver in situations where a patient's own body is unable to adequately control core temperature due to certain factors, such as disease, injury, anesthesia, open cavity surgery. Thus, apparatus 10 may provide input to the temperature management device to control the temperature and duration of the applied temperature based on the real-time feedback information determined by microcontroller 24.

The human body contains many cold and warmth receptors found primarily in the skin, spinal cord, abdominal viscera, and thorax. These receptors are responsible for providing temperature information from the body to hypothalamus and its numerous temperature-regulating centers. The main role of these temperature regulating centers is to detect abnormal body temperatures and to initiate body's response to either increase or decrease a body's temperature. In healthy individuals, there are three primary mechanisms that decrease body's temperature and three opposite mechanisms that increase body's temperature. The three mechanisms for decreasing temperature include: (1) Vasodilation of skin blood vessels, which results in increased blood flow, (2) sweating, and (3) decrease in body's heat production through inhibition of shivering and chemical thermogenesis, including the slowing of metabolic processes. In contrast, the body increases temperature through: (1) Vasoconstriction, which results in a decrease of blood flow, (2) piloerection, and (3) increase in chemical thermogenesis.

Based on the fact that primary temperature control mechanisms involve increase and decrease of blood flow, blood flow is, therefore, also a factor when evaluating a subject's response to temperature changes. Poiseuille's Law states that the fluid flow is proportional to the fourth power of the vessel radius. This means that very small changes in blood vessel radius will have profound effect on the blood flow though that vessel. Changes in blood flow, due to vasoconstriction or vasodilatation, will result in a change of delivered oxygenated hemoglobin and removed deoxygenated hemoglobin from the tissue. These oxy- and deoxy-hemoglobin changes may be detected using a near infrared spectroscopy system.

Near infrared spectroscopy systems exploit tissue scattering and absorption of visible and near-infrared light (hence near infrared spectroscopy, NIRS) to detect changes in oxygenated and deoxygenated hemoglobin levels at various tissue depths. The detection of changes in hemoglobin is accomplished using light sources with at least two different wavelengths (e.g., 650 nm and 900 nm). At these wavelengths, tissue chromophores are major absorbers of photons. (FIG. 2A). For example, increased concentration of oxygenated hemoglobin results in increased number of chromophores in tissue, which reduce the number of photons (at 900 nm wavelength) being detected on the surface of the tissue. Dependence (1) shows this inverse relationship between the concentration of chromophores in tissue and a number of detected photons on the tissue surface.

$${c}_{λ} ∝ \frac{1}{{n}_{λ}} \qquad ( 1 )$$

Based on the physiological temperature control mechanism of the human body and the detection capabilities of the NIRS system, the following are expected responses to thermal management therapy:

TABLE 1
Expected Physiological and NIRS System Responses to Thermal Management
Therapy

| | | | | NIRS System Response | |
|---|---|---|---|---|---|
| | Body Response | | | (number of | |
| | | Oxy | Deoxy | detected photons) | |
| | | Hemoglobin | Hemoglobin | 650 nm | 900 nm |
| Therapy | Blood Vessels | Concentration | Concentration | (deoxy) | (oxy) |
| Cooling | Vasoconstriction | ↓ | initially ↓ then | initially ↑ then | ↑ |
| | | | possibly ↑ | possibly ↓ | |
| Warming | Vasodilatation | ↑ | ↑ | ↓ | ↓ |

Table 1 shows that during the course of applying thermal therapy, such as a cooling thermal management therapy, blood vessels will constrict and cause a reduction in blood flow to monitored tissue. The reduction in blood flow to the tissue will cause a decrease in oxygenated hemoglobin concentrations resulting in detection of increased number of photons in the 900 nm region (dependence 1, above and FIG. 2A). In contrast, during warming thermal therapy, blood vessels will dilate causing an increase in blood flow to the monitored tissue, which will cause an increase in oxygenated hemoglobin concentrations resulting in detection of fewer number of photons in the 900 nm region. Therefore, based on the detection of photons in these regions, the microprocessor 24 can determine when continued treatment, cessation of treatment or a change in temperature is indicated, which can then be used to control the temperature management device.

Other remote devices that may be controlled by apparatus 10 include a support surface, for example, a medical mattress that provides medical treatment. Medical mattresses are often constructed as dynamic mattresses (e.g. mattresses with pneumatic bladders or fluidized beds) that can be adjusted to redistribute pressure to reduce the risk of a patient forming a pressure ulcer. In some cases, medical mattresses or toppers may have percussion or vibration devices that apply forces to the patient to help break-up the phlegm, for example, in patient's lungs. Other mattresses or toppers may incorporate stimulation devices, such as described in co-pending application Ser. No. 12/229,764 to Neustaedter, David et al. filed Aug. 27, 2008 entitled Apparatus for and method of preventing decubitis ulcers, which stimulate the patient to move themselves. Apparatus 10, therefore, may be coupled to the control system of a mattress or topper and provide input to the control system to provide an indication, for example, when a patient may need to be turned, receive percussion or vibration treatment or be stimulated to be moved. For example, the signals received by detector 22 may be analyzed to determine the level of oxygenation in the capillary bed of soft tissue, which can be indicative of the development of a pressure ulcer. Apparatus 10 may also include other sensors, such as a sensor to detect moisture and/or a sensor to detect temperature, which when combined with the oxygenation level information may provide a predictor of whether a subject is at risk of developing a pressure ulcer. When such risk is determined, then apparatus 10 may provide signals to the mattress control system to indicate that the patient should be turned, for example. Alternately or in addition, apparatus 10 may generate an alarm, such as a local alarm at the bed or an alarm through the bed communication system so that an alarm is sent to a nurse call system, for example. In addition, as noted, apparatus 10 may include a display, which is configured to display a map of the patient's interface with the mattress or topper that indicates regions where the risk of developing a pressure ulcer is elevated, for example, based on the oxygenation level information and other optional parameters (e.g., temperature and/or moisture).

Other remote devices that may receive input from apparatus 10 include reduced pressure therapy devices, such as vacuum assist devices (VACs), which are used in wound healing. A caregiver typically secures a VAC device to a subject's tissue around a wound, which applies a reduced pressure or vacuum to the wound, for one or multiple treatments until the wound is closed or at least partially closed. It has been shown that providing a vacuum or reduced pressure in proximity to a wound site accelerates the growth of new tissue at the wound. The negative pressure treatment increases capillary blood flow to the area and consequently faster formulation of granulation in the underlying tissue. Current dressings, however, can lead to maceration of the region around the wound. By monitoring the subject's tissue response to the negative pressure treatment, the negative pressure treatment may be adjusted automatically either in duration or pressure based on how the subject is responding. For example, apparatus 10 may be used to monitor the increased blood flow in the vicinity of the wound, inflammation, or to monitor granulation tissue formation to provide a measure of how the wound healing process is progressing or whether a VAC device is needed. The classic model of wound healing comprises three overlapping phases: inflammation, proliferation and remodeling. Within the first few minutes after the injury, a clot is formed, which reduces active bleeding. During the inflammation phase, bacteria and cell debris are removed from the wound by white blood cells. Blood factors are released into the wound that cause the migration and division of cells during the proliferative phase. The proliferation phase is characterized by angiogenesis, collagen deposition, granulation tissue formation, epithelialization, and wound contraction. By detecting that a wound is progressing normally, a clinician may decide that negative pressure treatment is not needed. On the other hand, the information collected by apparatus 10 may indicate negative pressure treatment is suitable and, further, as noted may provide feedback to the VAC device based on the patient's tissue response to treatment. For example, if during treatment, apparatus 10 detects that there is an increased blood flow to the monitored tissue area, apparatus 10 may provide input to the VAC device to terminate, continue treatment or adjust the pressure or may initiate an alarm to a caregiver to indicate to the caregiver that the VAC device should be turned off.

Further, in each case apparatus 10 may be incorporated into the treatment device itself to form part of a control feedback loop for the treatment device or may be simply in communication with the device either by hardwiring or wireless transmission or in communication with a caregiver through the display or through an alarm.

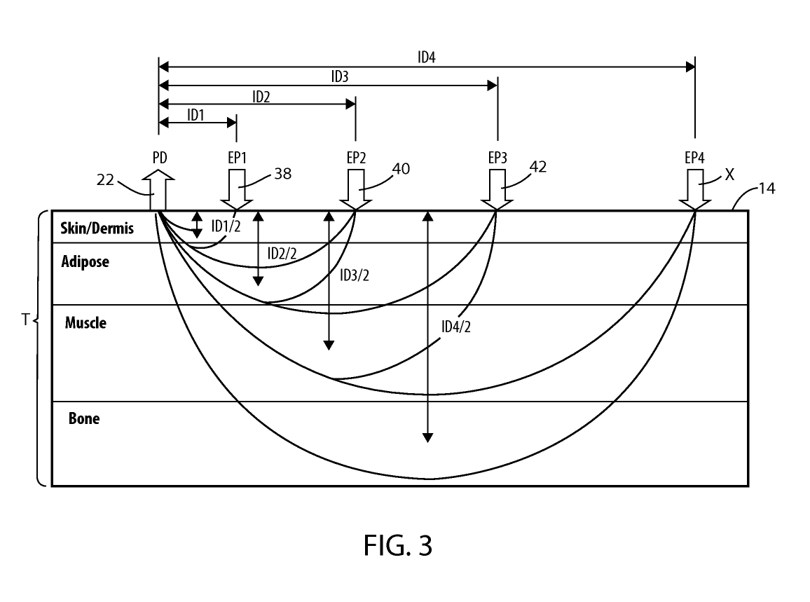

As described above, light source 16 may be supported on carrier 14, which may be adapted to be applied to a subject's tissue, such as skin. Further, as described above, light source 16 may have multiple light sources (16a-16f). Referring to FIG. 3, the light sources may be arranged in pairs 38 (e.g. 16a and 16d), 40 (e.g. 16b and 16e), 42 (e.g. 16c and 16f), and X. Each pair includes one light source emitting light at one frequency and the other light source emitting light at a second frequency different than the first frequency. For example, as noted, one frequency could fall in the red region of the light spectrum, and the other in the near infrared range of the light spectrum. Each pair 38, 40, 42 . . . X is spaced from detector 22 so that their incident light samples different tissue depth. As best seen in FIG. 3, the further the spacing the greater the depth of tissue sampled. Thus, apparatus 10 can be used to measure how chromophore levels vary in different tissue layers. The inter-optode distance (ID₁, . . . ID₄), between detector 22 and the light source pair determines the sampling depth. As noted, the distance between the light source(s) and the detector(s) determines extent of light penetration into the tissue and can extend up to about 3 cm in depth.

As noted above, carrier 14 may be formed from a flexible substrate with an adhesive backing or layer so that sensor assembly 12 may be placed on the subject's tissue and retained in place by the adhesive. Alternately, a strap or other holding device may be used to keep sensor assembly 12 in place. Further, carrier 14 may comprise a flexible substrate in the form of a film or transfer sheet so that the sensor assembly 12 may be transferred onto tissue and then left in place without the carrier.

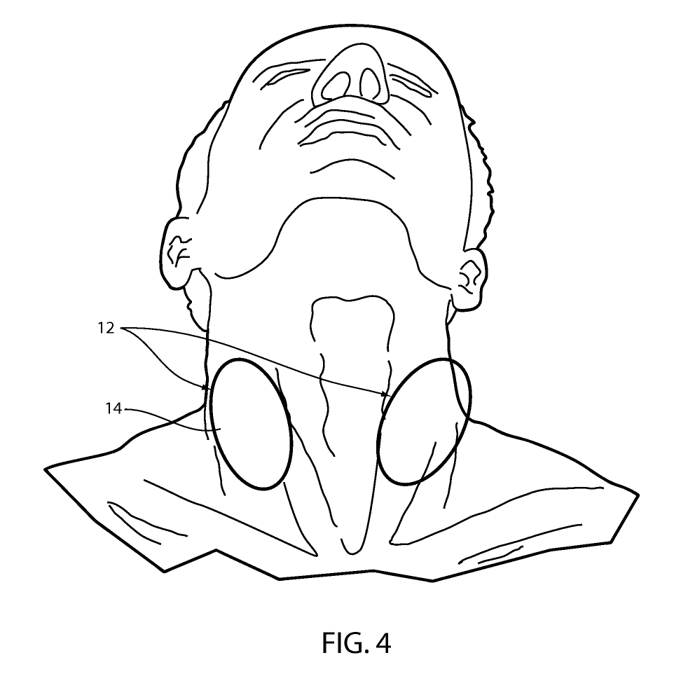

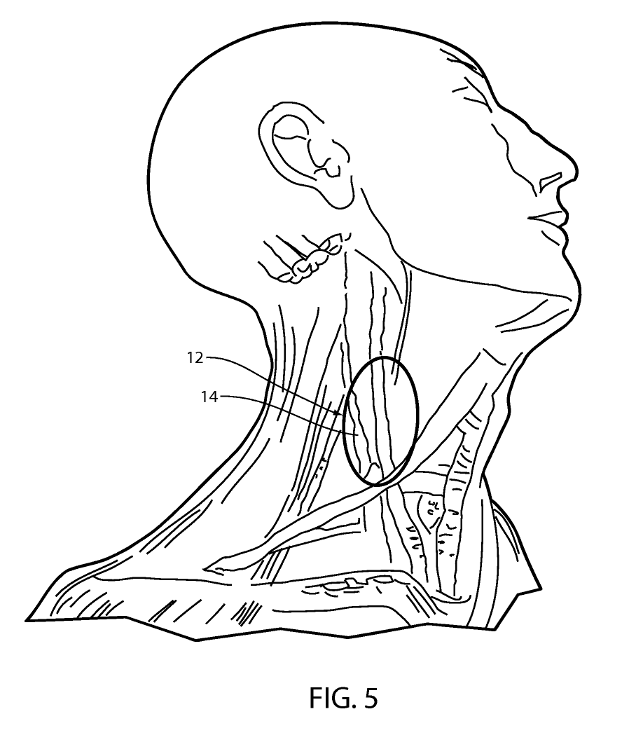

The location of sensor assembly 12 may vary. For example, referring to FIG. 4, sensor assembly 12 may be located on a subject's neck to detect changes in oxygenated and deoxygenated blood flowing from the heart to the brain or from the brain to the heart and lungs. Referring to FIG. 5, sensor assembly 12 may be applied to the skin and located over the common carotid artery (right side shown, but can also be applied to left side) to detect changes in oxygenated blood flowing from the heart to the brain. A second sensor assembly may be placed over the left side to monitor both left and right vascular structures. The assembly may also be placed over external and internal veins (e.g. internal Jugular vein and external Jugular vein) again to detect changes in deoxygenated blood flow from the brain to the heart and lungs. When several sensor assemblies are used, they may operate independently or they may be in communication with each other and have a shared microcontroller or may send signals to a central microcontroller, which processes the signals from the microcontrollers of all sensor assemblies.

Sensor assembly 12 may also be placed internally in a subject's body. For example, carrier 14 and/or sensor assembly 12 may be mounted to a stent, or carrier 14 may be configured as a stent. Similarly, carrier 14 and/or sensor assembly 12 may be mounted to a catheter. In this manner, tissue monitoring apparatus 10 may be used to monitor activity in a vascular structure, for example to monitor brain perfusion or to monitor other organs such as the heart, kidneys, lungs, or liver.

Thus, a tissue monitoring device and method are described that provide a non-invasive, lower power device and/or method for detecting changes in tissue state, for example, changes in tissue oxygenation levels, either as a result or internal factors or external factors, including treatment. The device and/or method can provide depth dependent information about a subject's tissue, which may be used to determine various parameters about the tissue state, including changes in perfusion and changes in the underlying tissue metabolism as it relates to these external and/or internal factors. This physiological information can be displayed or relayed or used to provide feedback information for the control of a remote device, such a treatment device, including a medical mattress.

As described, the present tissue monitoring device may consist of two components, a sensor assembly and a carrier, such as a disposable sensor strip. The sensor assembly may have one or more light sources and one or more detectors. The device may include a microcontroller and a wireless module for data transmission—or may be directly coupled to another device, such as a display or treatment device. It should be understood that alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. A tissue monitoring device for selecting, administering or adjusting a treatment based on information obtained by the monitoring comprising:
a substrate for applying to a tissue, for example, a human or an animal external, or internal tissue, including vascular tissue;
a light source supported by the substrate, the light source emitting light of a first frequency for directing the emitted light onto a surface of the tissue;
a photodetector carried by the substrate spaced from the light source for detecting scattered light, wherein the scattered light is the emitted light scattered by the tissue; and
a controller in communication with the photodetector and the light source.

2. The tissue monitoring device according to claim 1, wherein each of the emitted light and the scattered light has an intensity, the controller determining scattered light intensity with respect to the intensity of the emitted light.

3. The tissue monitoring device according to claim 1, wherein the light source comprises more than one light source.

4. The tissue monitoring device according to claim 1, wherein the light source further emits light at a second frequency, wherein the second frequency is different from the first frequency and optionally measuring the scattered light intensity to measure the absorption accrued in the tissue for each frequency.

5. The tissue monitoring device according to claim 1, wherein the light source comprises a discrete light source.

6. The tissue monitoring device according to claim 1, wherein the light source comprises a contiguous light source.

7. The tissue monitoring device according to claim 3, wherein each light source has an identity, and the controller correlates the identities with the detected scattered light intensity.

8. The tissue monitoring device according to claim 1, wherein the light source comprises one or more of an LED, a laser, or a plasma lamp.

9. The tissue monitoring device according to claim 1, wherein the photodetector comprises one or more photosensitive elements, such as a photodiode, a phototransistor, a photomultiplier, or a CCD.

10. The tissue monitoring device according to claim 1, wherein the controller is configured to monitor changes and status in intracellular and extracellular elements of the tissue.

11. A tissue monitoring system comprising:
a tissue monitoring device for selecting, administering or adjusting a treatment based on information obtained by the monitoring comprising:
a light source for directing emitted light into a tissue of a subject;
a photodetector spaced from the light source for detecting scattered light wherein the scattered light is the emitted light scattered by the tissue;
a remote device in communication with the tissue monitoring device; and
a controller in communication with the photodetector and with the remote device.

12. The tissue monitoring system according to claim 11, wherein the remote device comprises a treatment device for applying a treatment to a subject, and the controller in communication with the treatment device for adjusting the treatment based on the scattered light detected by the photodetector.

13. The tissue monitoring system according to claim 11, wherein the remote device further comprises a display, the display in communication with the controller and displaying information based on the scattered light detected by the photodetector.

14. The tissue monitoring system according to claim 11, wherein the remote device further comprises a data storage unit, the data storage unit in communication with the controller for storing information based on the scattered light detected by the photodetector.

15. The tissue monitoring system according to claim 11, wherein the controller comprises a local controller and a remote controller, the remote controller in communication with the local controller for analyzing the scattered light detected by the photodetector.

16. The tissue monitoring system according to claim 11, wherein each of the emitted light and the scattered light has an intensity, the controller or local controller determining scattered light intensity with respect to the intensity of the emitted light.

17. The tissue monitoring system according to claim 16, wherein the controller or local controller determines the scattered light intensity as a function of time.

18. A method of monitoring the effects of an internal or external factor on a subject's tissue, the method comprising:
emitting light of a first frequency;
directing the emitted light onto a surface of tissue; and
detecting scattered light intensity, wherein the scattered light intensity is the emitted light scattered by the tissue.

19. The method according to claim 18, wherein the directing includes directing light of more than one frequency, and optionally measuring the scattered light intensity to determine the absorption accrued in the tissue for each frequency.

20. The method according to claim 18, further comprising transmitting information about the scattered light to a caregiver or a remote device.

21. The method according to claim 18, further comprising controlling a remote device based on the determining of the scattered light intensity.

22. The method according to claim 21, further comprising administering a treatment based on the scattered light intensity, adjusting the administering of a treatment based on the scattered light intensity, or displaying information based on the scattered light intensity.

23. The method according to claim 22, wherein the adjusting of the administering of a treatment includes adjusting an application of thermal therapy or reduced pressure therapy or vibration/percussion therapy or drug therapy.

24. The method according to claim 22, wherein the determining the scattered light intensity includes determining the light intensity as a function of time.

25. The method according to claim 18, further comprising monitoring the scattered light intensity continuously or periodically, and optionally using information about the scattered light intensity to identify trends in a physiological condition of the subject.
